(19) United States
(12) Patent Application Publication
(10) Pub. No.: US 2010/0137814 A1
(43) Pub. Date: Jun. 3, 2010

(54) ATRAUMATIC EAR IRRIGATION DEVICE

(75) Inventor: Heng Hai Chew, Permatang Pauh (MY)

Correspondence Address: BUTZEL LONG;IP DOCKETING DEPT, 350 SOUTH MAIN STREET, SUITE 300, ANN ARBOR, MI, 48104, US

(73) Assignee: Mecha-Medic Solution Sdn. Bhd., Bayan Lepas (MY)

(21) Appl. No.: 12/064,979

(22) PCT Filed: Sep. 21, 2006
(86) PCT No.: PCT/MY06/00011

§ 371 Date: Feb. 27, 2008

(30) Foreign Application Priority Data
Sep. 22, 2005 (MY) PI2005-4427

(51) Int. Cl. A61M 3/02

(52) U.S. Cl. 604/257

(57) ABSTRACT

An ear irrigation device for delivering fluid into the auditory canal (2) for removal of earwax (3), which is the entire ear piece (1), when in use to be fitted into an ear canal (2), that has a longitudinal extended body of hollow tubing (4) coiled as a helical spring with its distal end, as the fluid outlet (7), to be positioned, when in use, proximate to the inner portion of the external ear and the proximal end, as the fluid inlet (5), for attaching to a fluid delivery means (6), such as a syringe or a pump, on the outside.

### FIELD OF INVENTION

The present invention relates to ear irrigation and in particular to a self ear irrigation device for removing ear wax from the external auditory canal.

### BACKGROUND OF THE INVENTION

Ear wax is normal product of the ear which protects the skin of the ear from water and infection. Ear wax is formed from wax glands in the external ear canal as well as other components such as dead skin, sweat and oil. The primary component of ear wax is keratin (derived from dead skin). Ear wax may also include cerumen, which is the secretory product of the ceruminous glands in the external ear canal Ear wax can plug up the ear thereby causing hearing to be reduced, and a full feeling in the ear. It can also trap bacteria in the ear leading to infection. Ear Irrigation or “syringing” is a standard and accepted method of wax removal. However there is always a danger of perforation of the ear drum and with such a perforation, irrigation may force water and wax into the middle ear, causing a nidus for infection.

Ear Irrigation is generally performed by doctors, nurses or caregivers to remove a build up or accumulation of earwax or cerumen within the external auditory canal.

This is done usually by involving the injection, under pressure, of a cleaning fluid or water into the canal, through an ear irrigation device, using a syringe or pump that squirts the cleaning fluid or water, into the ear cavity, to dilute and remove the ear wax within. The fluid or water then circulates inside the canal and then drains out of the ear carrying with it the ear wax.

Though the procedure is relatively simple, it should preferably be handled by an expert or professional, although it can be done, just as well, by a non expert or even self administered if a proper device is used, otherwise it would be a more traumatic experience for the patient.

There are various ear irrigation devices which varies from the more sophisticated ones that require an expert or professional to operate to the simple ones that can be self applied, but they normally comprise of a longitudinal hollow tubing or an earpiece that is inserted into the ear at one end and at the other attached to a fluid delivery means to provide a continuous fluid flow, in the form of a syringe or pump to administer the cleaning fluid which could be just saline solution. The ear piece usually comprises of a flexible plastic tubing that has a longitudinal shape with its distal end extending inside the ear and the proximal end attached to the syringe or pump for the delivery of the fluid.

Care should be used to ensure that tip of the ear piece or the impact of the in jet of the fluid does not hurt the eardrums and at the same time allowance should be made for the fluid to flow out freely together with the dislodged ear wax.

Prior art devices found in U.S. Pat. No. 4,206,756, U.S. Pat. No. 5,364,343 and U.S. Pat. No. 6,210,358 where they all generally try to address the problem with attempting to minimize the impact on to the ear drum by the projected fluid, when it strikes the tympanic membrane (ear drum), for example in U.S. Pat. No. 5,364,343 an attempt is made by applying means to divert the flow away from the centre, to project fluid inwardly towards the wall surfaces, but invariably the impact of the fluid may still cause damage or discomfort to the patient.

Furthermore, the other problem is, unless adequate means is provided for the fluid to freely flow out of the ear together with the ear wax, it may result in pushing the ear wax further into the ear canal, thereby aggravating the problem by creating a further build up of pressure in the blocked auditory canal which will cause more pain, discomfort and a really traumatic experience. For example in both U.S. Pat. No. 5,364,343 and U.S. Pat. No. 6,210,358 attempts are disclosed to provide for an outlet for the outflow of the fluid and ear wax, however the outlet being itself too narrow, may result in a total occlusion of the outlet which can lead to increased pressure inside the auditory canal that may result in a rupture of the tympanic membrane.

There is therefore a continuous need for a simple, easy to use, ear irrigation device that can efficiently remove the accumulation of the ear wax inside the auditory canal without causing any discomfort or traumatic experience to the patient, and which can be administered by the patient without the need for any professional help.

It is therefore the primary object of this invention to provide for atraumatic ear irrigation device that is also safe to use by the patient or any non-professional and yet can satisfactorily overcome the aforesaid problems found in prior art devices.

It is another object of this invention to provide for such a device that is also cheap to manufacture, simple and yet effective.

It is yet another object of this invention to provide for a device that is truly safe and atraumatic, especially when it is self-administered by the patient.

### SUMMARY OF THE INVENTION

In accordance to the present invention, there is provided an ear irrigation device for delivering fluid into the auditory canal, which is the entire ear piece, when in use to be fitted into ear canal, that has a longitudinal extending body of hollow tubing coiled as a helical spring with its distal end, as the fluid outlet, to be positioned, when in use, proximate to the inner portion of the external ear and the proximal end, as the fluid inlet, for attaching to a delivery means, such as a syringe or a pump, on the outside.

The present invention provides for an outflow of water that continues to circulate until it reaches the end at the tympanic membrane, thereby reducing the impact of the water flow and at the same time providing a more efficient flush.

The ability of the spring portion to compress itself and accommodate the structure and the length of the ear canal, will not only prevent the ear wax to be pushed further inwards thereby aggravating the problem but also will not cause pain and discomfort to the patient.

The middle of the spring structure will serve as a very wide outlet for the water to flow out freely from the auditory canal without any danger of obstruction.

In order for the invention to be fully understood, the invention will now be described with reference to the accompanying drawings.

### BRIEF DESCRIPTION OF THE DRAWINGS

FIG. 1 is a diagrammatic representation of the present invention in use.

FIG. 2 is a diagrammatic representation illustrating the spiral flow of the cleaning fluid squirting out from the fluid outlet of the helical coiled spring of the present invention till the tympanic membrane and the outflow from the ear canal.

FIG. 3 is a diagrammatic representation showing the compressible helical coiled spring of the present invention accommodating to the buildup of ear wax in the ear canal.

FIG. 4 is a diagrammatic representation illustrating the removal of the buildup of the ear wax by spiral flow of the cleaning fluid in different stages.

FIG. 5 is a diagrammatic representation illustrating the flexibility of the helical-coiled spring of the present invention accommodating the shape and curvature of the external auditory canal.

FIG. 6 is a cross-sectional view of a ear with the present invention inserted in it when in use.

FIG. 7 is a diagrammatic representation of the present invention illustrating the removal of foreign body lodged inside the ear canal.

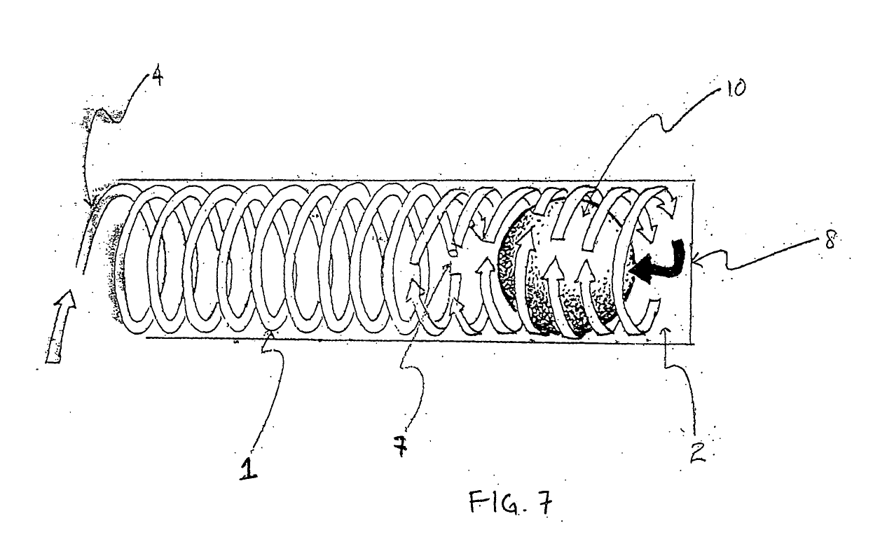

### DETAILED DESCRIPTION OF A PREFERRED EMBODIMENT

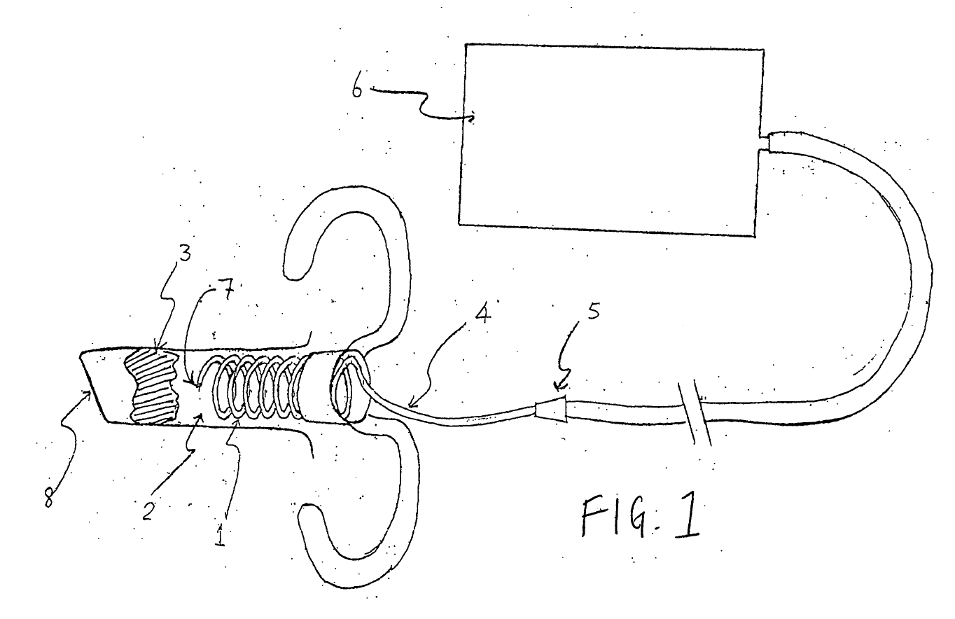

Referring to FIG. 1 is disclosed an ear irrigation device which essentially is an ear piece (1) that fits into the ear canal (2) for irrigating and removal of ear wax (3) comprising of a longitudinal hollow tubing (4), made of a flexible material preferably a thermoplastic, that is coiled as a helical spring preferably throughout its entire length of that portion which is inserted in the auditory canal (2) with its proximal end acting as the fluid inlet (5), made to attach on to a fluid delivery means (6), preferably a syringe and the distal end, acting as the fluid outlet (7), which is when in use and inserted into the auditory canal (2), positioned proximate to the inner portion of the external ear canal (2) a safe distance away from the tympanic membrane (8).

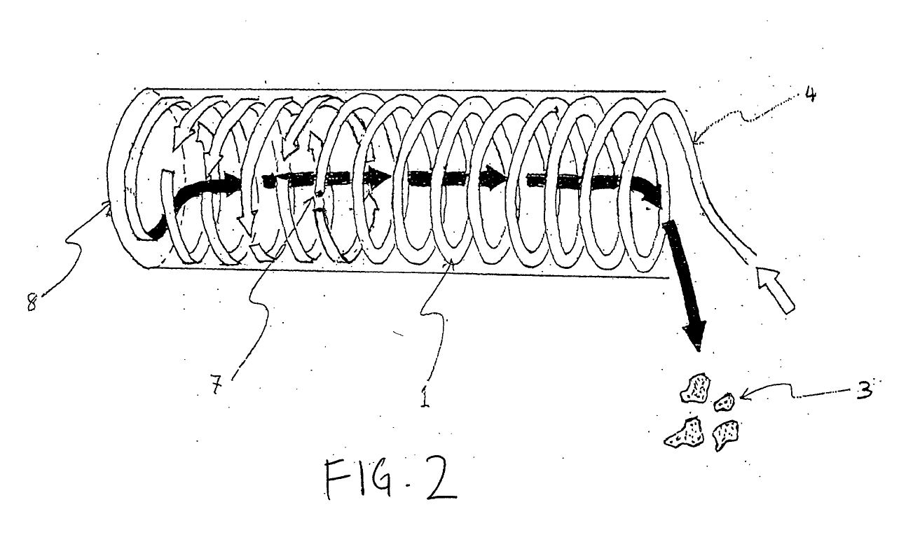

Referring to FIG. 2, the water or cleaning fluid which is squirted out through the fluid outlet (7) would continue to flow in spiral manner, flushing the inner ear canal (2) gently in a circular motion until it reaches the tympanic membrane (8), without causing any direct impact.

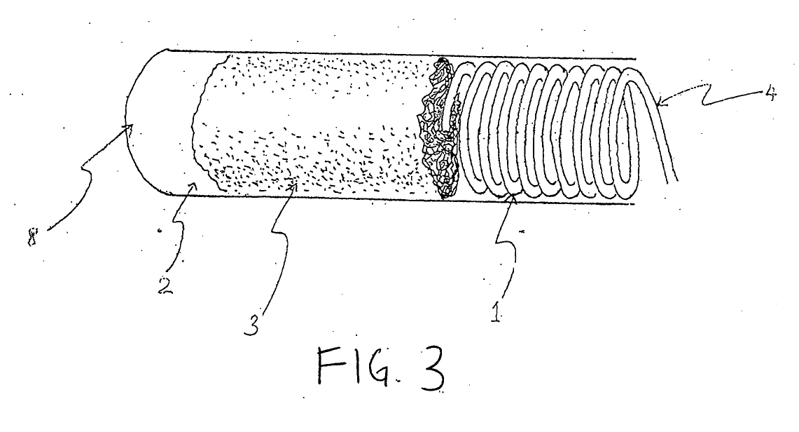

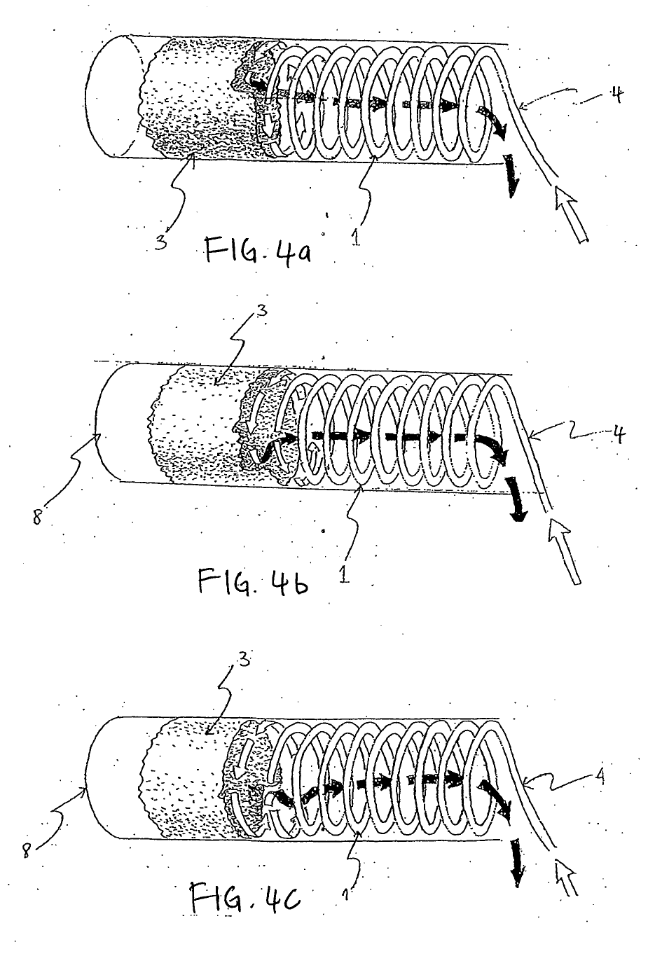

Thereafter the excess water will flow outwards through the centre of the helical spring unrestricted, carrying along with it any dislodged earwax or cerumen (3). If, as shown in FIG. 3, where there is a build up of earwax (3), then the helical spring structure of the ear piece (1) will compress itself to a shorter length and as shown in FIGS. 4 (a), (b), (c), the circular motion of the flushing water will wedge itself into the earwax (3) and will break the accumulation of earwax (3) thereby removing it eventually by burrowing through it and scraping the edge of the ear canal (2), instead of merely pushing it further inwards, further aggravating the problem.

As already been illustrated in FIGS. 4 (a), (b), (c), the spiral flow of the water removes the accumulation of the earwax (3) lodged in the ear canal (2) with the jet impact of the water on the earwax (3) at a consistent position which shifts itself gradually inwards into the ear canal (2) as the earwax (3) dislodges, ultimately dislodging the entire build up of earwax (3). This is compared to the prior art devices which only would impact a consistently fixed position on the wall of the ear canal (3) irregardless of where the earwax (3) is actually lodged or in circumstances in which the ear canal (2) may actually be already cleared and the ear wax dislodged, thereby not really performing an efficient job unless they are manually monitored and adjusted under vision with a supplementary viewing device.

Besides, the present invention can also easily accommodate the use of an ordinary supplementary viewing device, in the event if it is required, by inserting the said device into the middle hole of the helical spring.

Thus it is clear that the present invention with its helical spring structure can effectively and efficiently remove all accumulation of earwax (3) from the auditory canal (2) faster, with lesser liquid and utilizing lesser effort than any prior art devices.

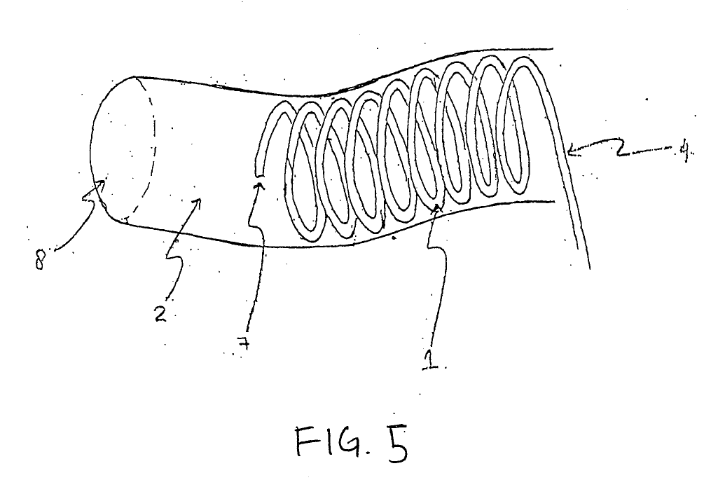

The helical spring structure of the ear piece (1) has the ability to adjust itself in accordance to the length of the ear canal (2) as shown in FIG. 2 and to accommodate any build up of the earwax (3), as well as bends and twists and adjusts itself to take in the shape, curvature and length of the auditory canal (2) as shown in FIG. 5, unlike the rigid structures of the prior art devices, thereby eliminating any discomfort, pain or trauma to the patient.

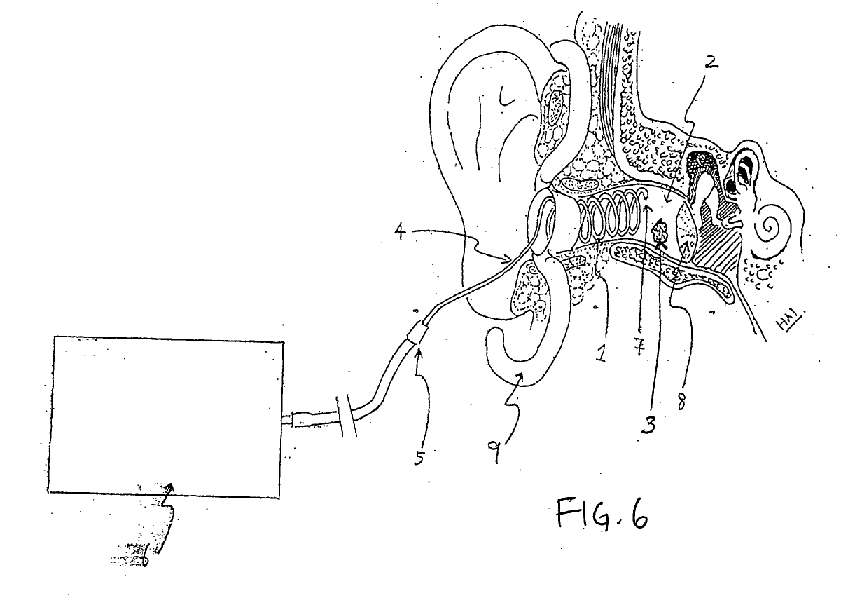

The present invention as shown in FIG. 6 can also have a built-in ear attachment means (9) comprising of preferably two mirror image hooks that can enable the device to be secured onto either of the outer ear.

The present invention can be also utilized to remove and dislodge other particles or foreign bodies (10) that may be inadvertently lodged in the auditory canal (2) as shown in FIG. (7).

1. An ear irrigation device that comprises of an earpiece (1) which fits in the ear canal (2) that is made up of a longitudinal hollow tubing (4) that is coiled as a helical spring with its fluid inlet (5) at the proximal end and attached to a fluid delivery means (6) and its fluid outlet (7) at the distal end which is when in use and inserted into the auditory canal (2) positioned proximate to the inner portion of the external ear canal (2) a safe distance away from the tympanic membrane (8).

2. An ear irrigation device as claimed in claim 1 wherein the longitudinal hollow tubing (4) is made of a thermoplastic material.

3. An ear irrigation device as claimed in claim 1 wherein the fluid delivery means (6) is a syringe.

4. An ear irrigation device as claimed in claim 1 wherein the helical coil spring runs the entire length of the ear piece (1).

5. An ear irrigation device as claimed in claim 1 wherein it further comprises of a built-in outer ear attachment means (9).

6. An ear irrigation device as claimed in claim 5 wherein the outer ear attachment means comprises of two mirror-image hooks.
